(19) United States
(12) Patent Application Publication
(10) Pub. No.: US 2016/0139096 A1
(43) Pub. Date: May 19, 2016

(54) Method And Apparatus Determining An Amount of Phosphine (PH3) In An Atmosphere of An Enclosed Area

(71) Applicant: Dennis John Glennon, Warwick, RI (US)

(72) Inventor: Dennis John Glennon, Warwick, RI (US)

(21) Appl. No.: 14/940,533

(22) Filed: Nov. 13, 2015

Related U.S. Application Data
(60) Provisional application No. 62/081,344, filed on Nov. 18, 2014.

(51) Int. Cl. G01N 33/00; H04L 29/06; G01N 29/24; H04L 29/08; G01N 21/3504; G01N 29/036

(52) U.S. Cl. CPC G01N 33/006; G01N 21/3504; G01N 29/036; G01N 29/2418; H04L 67/32; H04L 63/0428

(57) ABSTRACT

A process is provided for determining the amount of phosphine gas in the atmosphere of an enclosed area that has been fumigated with phosphine (PH₃). This process comprises: (A) sampling said atmosphere of said enclosed area to obtain a gaseous sample; (B) selectively removing water from said gaseous sample by passing said gaseous sample through an evaporation zone to obtain a dry sample, wherein said evaporation zone comprises a copolymer of tetrafluoroethylene and perfluoro-3,6-dioxa-4-methyl-7-octene-sulfonic acid; and (C) analyzing said per-evaporated sample in a detector to determine the amount of phosphine gas in said dry sample. The process allows for heretofore unattained capability of monitor measurements of phosphine gas (PH₃) up to 1% (10,000 ppm).

### CROSS REFERENCE TO RELATED APPLICATIONS

The present application claims the benefit of U.S. Provisional Patent Application No. 62/081,344, filed on Nov. 18, 2015, which is incorporated herein by reference in its entirety.

### BACKGROUND

Phosphine is manufactured by the hydrolysis of metal-phosphides, by the electrolysis of phosphorus in the presence of hydrogen, and by a phosphorus-steam reaction. The most common preparation of phosphine for fumigation is the hydrolysis of metal phosphides as shown here:

(AlP or Mg₃P₂ or Ca₃P₂)+3 H20→PH₃+Al(OH)₃ (Metal phosphides) Phosphine (gas)+Metal Hydroxide

There are multiple commercially available metal phosphide products. A partial list is provided.

Commercial Metal Phosphides (Partial List)

Alutal
Celphide (tablets)
Celphine (tablets)
Celphos (tablets)
Delicia Gastoxin
Detia Gas-Ex-B (bags)
Detia Gas-Ex-P (pellets)
Detia Gas-Ex-T (tablets)
Phosfume (pellets and tablets)
Phostoxin (pellets, tablets, “prepacs”, strips)
Quickfos (pellets and tablets)
Detiaphos (pellets)
Magtoxin (pellets, tablets, rounds)

There are also commercial sources of cylinderized phosphine both as dilute (approximately 2% PH₃/balance CO₂) and approaching 100% (approximately 98-99% PH₃).

Pure phosphine has an auto-ignition temperature of 38° C. but, because of the presence of other phosphorus hydrides (particularly diphosphine) as impurities, the technical product often ignites spontaneously at room temperature (ACGIH, 1986). Phosphine forms explosive mixtures with air at concentrations greater than 1.8% (18,000 ppm, approx. 25,000 mg/m³ as lower explosion limit). Oxidation of phosphine involves a branching chain reaction in air, the upper and lower explosion limits of phosphine depends on the temperature, pressure, and proportions of phosphine, oxygen, inert gases and non-condensed water vapor present. Higher Relative Humidity (RH) results in more oxidizing conditions for the phosphine gas.

Phosphine in the presence of air having non-condensed water (water vapor) will easily react to form various acids such as orthophosphorous acid (H₃PO₂), phosphorous acid (H₃PO₃) and phosphoric acid (H₃PO₄). These in turn will undergo further chemical reactions of degradation. This naturally occurring process is called oxidation.

The phosphorus in phosphine will with water vapor be oxidized from the −3 valence state to higher oxidation states at the gold coating layer (optical conduit) of any Non-Dispersive Infrared (NDIR) detector according to the following reactions:

P oxidized from −3 to +1 (catalyzed by gold (Au)) PH₃+2H₂O=H₃PO₂+4H++4e−(orthophosphorous acid)

P oxidized from −3 to +3 PH₃+3H2O=H₃PO₃+6H++6e−(phosphorous acid)

P oxidized from −3 to +5 PH₃+4H2O=H₃PO₄+8H++8e−(phosphoric acid)

The half-life of phosphine in air is about 28 h. The eventual oxidation product of phosphine will be phosphorus oxyacids and inorganic phosphate as a solid deposition.

The present invention, regardless of the source of phosphine used, eliminates relative-humidity contributing factors to oxidation of phosphine allowing NDIR technology an extended measuring range for phosphine to 1% (10,000 ppm).

### SUMMARY

Phosphine has several uses, including use as a fumigant. Fumigation is the use of certain gases to control insects and other pests. Phosphine has been used since the early thirties in various fields of pest control especially for disinfestation of grain in bags or bulk. Phosphine fumigation of agricultural commodities on board ship during transit is common practice in some parts of the world. Millions of tons of grain undergo a treatment with phosphine fumigation each year.

During fumigation it is important to know the amount of phosphine in the air of the enclosed area. This is because a certain level of phosphine is needed in the air of the enclosed area in order to rid such area of the insects and other target pests that are present therein.

It is an object of this invention to provide a phosphine fumigation process which has NDIR sensors or PAS sensors of extended life cycles used over an extended range (up to 1% PH₃; 10,000 ppm) with negligible cross sensitivity to CO2. A PAS sensor is an NDIR sensor if no dispersive optics are used, if, in place of dispersive optics such as a prism or grating a band pass filter is used, then it is an NDIR. Traditional NDIR measures the attenuation of IR energy with a thermal detector. The key difference between NDIR and PAS is how the absorbance of optical energy is measured. PAS measures pressure modulation with a microphone. The gas molecules absorb some of the light energy and convert it into an acoustic signal which is detected by a microphone. The chopper is a slotted disk that rotates and effectively “switches” the light on and off. The optical filter is a narrow-band IR interference filter. As the gas absorbs energy, it is heated and therefore expands and causes a pressure rise. As the light is chopped, the pressure will alternately increase and decrease—an acoustic signal is thus generated. The acoustic signal is detected by two microphones, hence the term photoacoustic infrared.

In accordance with this invention a process is provided for determining the amount of phosphine in the atmosphere of an enclosed area that has been fumigated with phosphine (PH3). This process comprises sampling an atmosphere of an enclosed area to obtain a gaseous sample; selectively removing water from the gaseous sample by passing the gaseous sample through an evaporation zone to obtain a per-evaporated sample, wherein the evaporation zone comprises a copolymer of tetrafluoroethylene and perfluoro-3,6-dioxa-4-methyl-7-octene-sulfonic acid; and analyzing the per-evaporated sample in a phosphine non-dispersive infrared (NDIR) detector to determine the amount of phosphine in the per-evaporated sample. Additional information on this invention is provided in detail in the following.

The detector is configured with a wavelength specific filter (e.g. a narrow bandpass filter) whose optical coating is optimized for the exclusion of water vapor and carbon dioxide (CO2) allowing accurate phosphine measurements. The realization of the above allows for heretofore unattained capability of NDIR measurements of phosphine gas (PH3) up to 1% (10,000 ppm).

### BRIEF DESCRIPTION OF THE DRAWINGS

The foregoing will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 depicts a block diagram of a Non-Dispersive Infrared Monitor in accordance with a particular embodiment of the present invention.

FIG. 2 is a block diagram of a PAS monitor in accordance with a particular embodiment of the present invention.

FIG. 3 is a block diagram of the secure data flow in accordance with a particular embodiment of the present invention.

FIG. 4 depicts a first embodiment of a method for a Non-Dispersive Infrared Monitor in accordance with a particular embodiment of the present invention.

FIG. 5 depicts a second embodiment of a method for a Non-Dispersive Infrared Monitor in accordance with a particular embodiment of the present invention.

FIG. 6 depicts a first embodiment of a method for secure data flow in accordance with a particular embodiment of the present invention.

### DETAILED DESCRIPTION

The embodiments set forth below represent the necessary information to enable those skilled in the art to practice the invention and illustrate the best mode of practicing embodiments of the invention. Upon reading the following description in light of the accompanying figures, those skilled in the art will understand the concepts of the invention and recognize applications of these concepts not particularly addressed herein. It should be understood that these concepts and applications fall within the scope of the disclosure and the accompanying claims.

The preferred embodiment of the invention will now be described with reference to the accompanying drawings. The invention may, however, be embodied in many different forms and should not be construed as limited to the embodiment set forth herein; rather, this embodiment is provided so that this disclosure will be thorough and complete, and will fully convey the scope of the invention to those skilled in the art. The terminology used in the detailed description of the particular embodiment illustrated in the accompanying drawings is not intended to be limiting of the invention. In the drawings, like numbers refer to like elements.

Fumigation is well known in the art. It can be conducted in any area that is enclosed or enclosable. Specific examples are silos for the storage of grains, ships for transit of commodities, shipping containers for in-transit and quarantine applications. For the purposes of this invention, the fumigant of choice is phosphine (PH₃). Phosphine's use as a fumigant for identified target pest requires extended application times compared to other fumigants. It is this requirement for lethality that demands new methods to address a well-documented world-wide resistance of pests to sub lethal dosages of phosphine fumigant gas concentrations realized in part to inaccurate measurements of this phosphine gas. Required accurate measurements of phosphine gas concentrations over time (CxT vales) have not been routinely attained. This invention provides a solution to this deficiency.

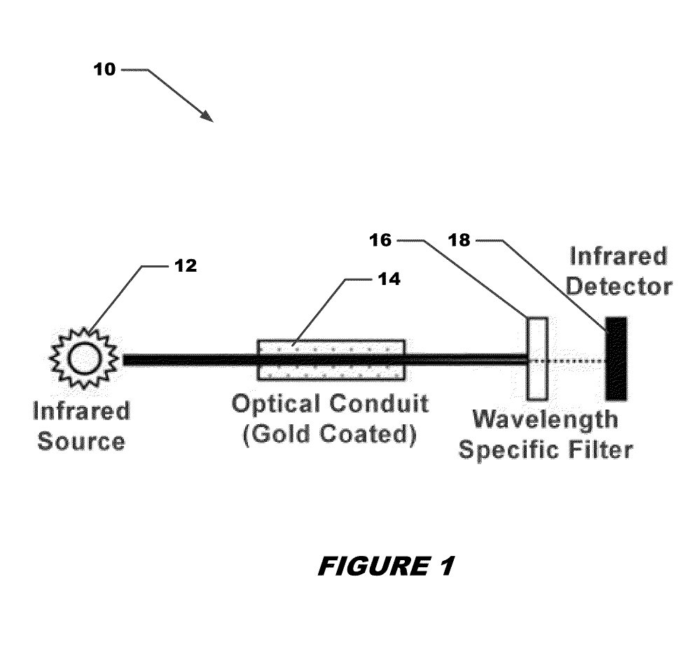

A Non-Dispersive Infrared (NDIR) Phosphine Monitor can be used to determine and accurately quantify an amount of phosphine gas in a fumigation enclosure. The NDIR monitor was invented and first used in Germany in the 1930's. For these many years since the infrared absorption of gases have been used for quantitative analysis of various gases. Regardless of the design and target gas they all have the same basic design. As shown in FIG. 1, an NDIR mo0nitor includes an infrared source 12, an optical conduit of highly reflective, non-absorbing coatings (gold) 14 and a wavelength specific optical filter 16 integrated with an infrared detector 18.

Monitors are available in different configurations all of which use well established Non-Dispersive Infrared Technology (NDIR) as the means of phosphine gas quantification. These portable/trans-portable instruments quantitatively measure the phosphine gas concentration in air by actively extracting air samples of the enclosed area. However, there are certain problems with using a Non-Dispersive Infrared Monitor for phosphine detection in the presence of atmospheric or elevated levels of water vapor. Non-Dispersive Infrared Technology uses certain and varied infrared light sources through a highly reflective conduit to deliver measured energy levels to different detector configurations. These NDIR Sensors all utilize enclosed reflective conduits constructed of precisely machined non-absorbing reflective metal layers principally gold (Au) coatings. The maintained integrity of these gold coatings allows for efficient, quantitative transfer of infrared energy levels from source to detector. It is this efficient transfer of energy that allows for absolute laws of physics to be employed in the quantification of phosphine gas.

These chemical reactions (oxidation of phosphine) and resulting products taken in aggregate will depending upon the resulting concentrations cause chemical oxidation of the gold conduit metal layer of the NDIR Sensor. The result of this natural, uncontrolled chemical oxidation of the gold surface is a pitting of the surface with resulting inefficiency of sensor operation and ultimate failure.

The rate of the sensor degradation and failure is dependent upon this pronounced compromise of the gold coated sensor conduit and its' ability to promote the transfer of infrared energy. That is, the amount of water vapor if uncompensated for, in the presence of phosphine adversely affects the performance and life expectancy of any sensor whose design is for the quantification of phosphine gas.

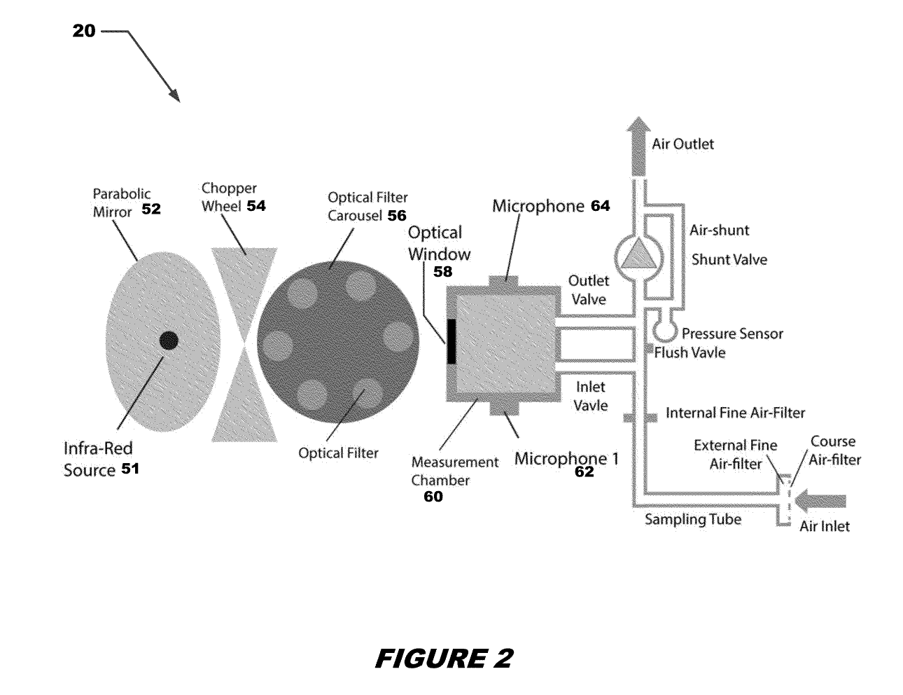

Another type of monitor which can be used is a Photoacoustic Spectroscopy (PAS) monitor shown as 50 in FIG. 2. In a PAS monitor the gas to be measured is irradiated by modulated light of a pre-selected wavelength. The gas molecules absorb some of the light energy and convert it into an acoustic signal which is detected by a microphone. The IR-source is a spherical, heated black body 51. A mirror 52 focuses the light onto the window 58 of the PAS cell (also referred to as a measurement chamber) after it has passed the light chopper 54 and the optical filter 56. The chopper 54 is a slotted disk that rotates and effectively “switches” the light on and off. The optical filter 56 is a narrow-band IR interference filter.

After passing through the window, the light beam enters the PAS-cell. If the frequency of the light coincides with an absorption band of the gas in the cell, the gas molecule will absorb part of the light. The higher the concentration of gas in the cell, the more light will be absorbed. As the gas absorbs energy, it is heated and therefore expands and causes a pressure rise. As the light is chopped, the pressure will alternately increase and decrease—an acoustic signal is thus generated. The acoustic signal is detected by two microphones 62 and 64. The electrical output signals from the two microphone signals are added in an amplifier, before they are processed.

The first step in this invention is to sample the atmosphere of the phosphine-fumigated-enclosed area to obtain a gaseous sample. This sampling can be conducted continuously or intermittently at the discretion of the licensed fumigation operator.

The second step in the process is to selectively remove non-condensed water from the gaseous sample. This is accomplished by passing the gaseous sample through an evaporation zone. This produces an evaporated sample. The evaporation zone comprises a membrane made from a copolymer of tetrafluoroethylene and perfluoro-3,6-dioxa-4-methyl-7-octene-sulfonic acid. Currently, it is preferred to use Nation® copolymer because it is highly selective in the removal of water from gases (Nation® is a registered trademark of E.I. DuPont). The sole supplier of Nation tubing to the world is PermaPure Inc. which has a web site at www.permapure.com. As the sample moves through the evaporation zone, water is selectively removed from the sample. The non-condensed water, so removed, then passes through the membrane wall of the copolymer where it evaporates into the surrounding air.

The third step of the process is to analyze the evaporated sample in a detector to determine the amount of phosphine in the evaporated sample. This analysis is conducted by a phosphine NDIR sensor or a PAS sensor whose integrity and hence proper operation is compromised if phosphine and non-condensed water are present in the NDIR sensor together at the same time. The continuous quantitative removal of this water vapor and resulting dry phosphine gas sample ensures accurate phosphine gas concentration readings and sensor integrity. One example of a phosphine monitor with active non-condensed water vapor removal is the PM400 and PM100 Series NDIR Monitors. The PM400 and PM100 NDIR Monitors are provided by Spectros Instruments, Inc., 17D Airport Road, Hopedale, Mass. 01747. After the amount of phosphine is determined, this value can be used to determine if a sufficient quantity of phosphine is present in the air and affect sufficient lethality for the target pest so that the enclosed area is properly fumigated.

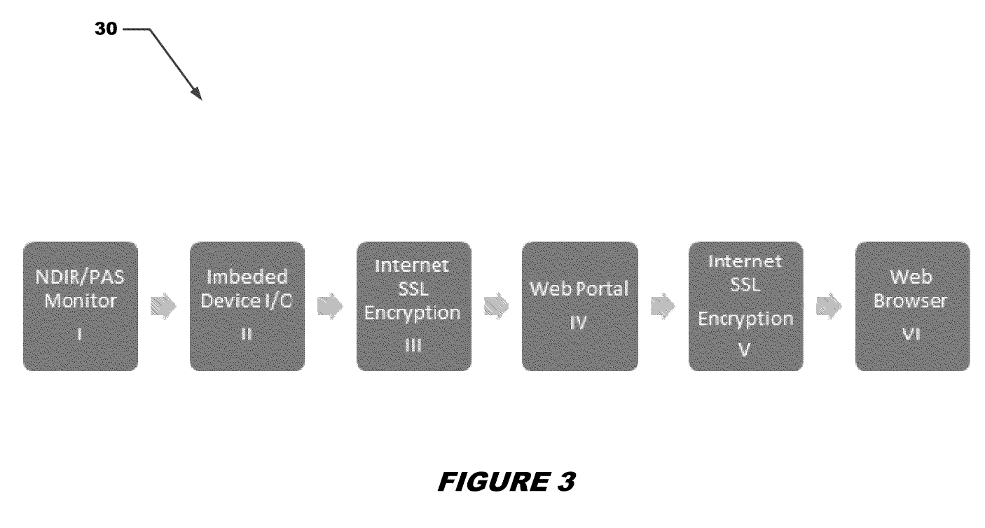

Referring now to FIG. 3, a process for secured data flow is shown. The obtained phosphine gas concentrations values are continuously logged with corresponding onboard sensor values for each reading. This involves sensor values from onboard Pressure and Temperature (P&T) sensors measuring the gas cell/chamber in NDIR and PAS Monitors. The measurements or readings are continuously confirmed to be within predefined firmware ranges for Gas Cell Voltage, Monitor Box Temperature, Detector Temperature, Gas Cell Pressure, Pressure Differentials between Gas Cell Pressure and Ambient Pressure all within electronic closed loop feedbacks of the NDIR and PAS Monitors.

Any phosphine gas concentration reading found to be outside of the required sensor values in the firmware is invalidated and the NDIR/PAS Monitors will return a fault condition rendering the reading null and void. These validated phosphine gas concentration readings are along with their associated sensor values continuously uploaded to a secured web database.

This process is buffered with onboard monitor data storage in the event of the loss of an internet connection. This also allows automatic sending of buffered data with reestablishment of internet connection that is encrypted and secure.

These phosphine gas concentrations obtained along with their recorded onboard sensor physical parameters (P,V,T) are validated individually and collectively to Absolute Law of Physics (Boyles Law, Charles Law, Avogadro's Law and all combined as Ideal Gas Law). This data is correct and will withstand technical and legal challenges. These validated phosphine gas concentrations allow the phosphine fumigation to be traceable to compliance with the Code of Federal Regulations that establishes the United States Food and Drug Administration (FDA) regulations on electronic records and electronic signatures. Title 21 CFR Part 11 Section 11.1 (a) details the criteria with electronic records and trustworthiness.

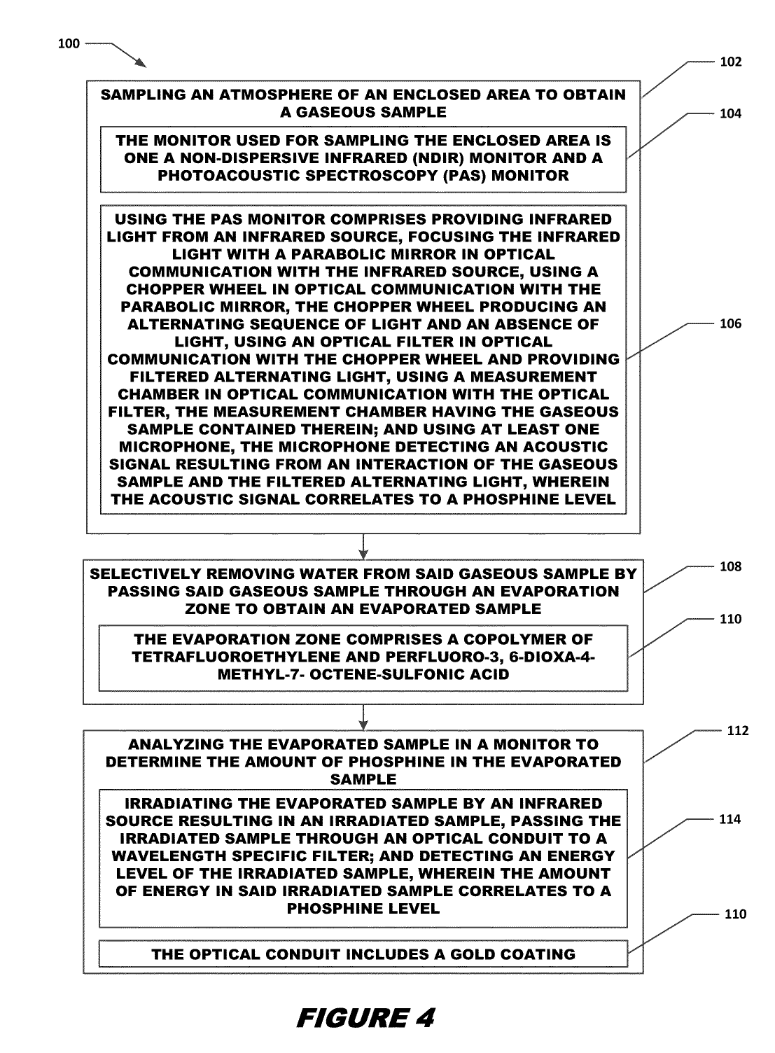

FIG. 4 is a flow diagram of a first method for determining an amount of phosphine in an enclosed area. Method 100 starts with processing block 102 which discloses sampling an atmosphere of an enclosed area to obtain a gaseous sample. As shown in processing block 104 the monitor used for sampling the enclosed area is one a Non-Dispersive Infrared (NDIR) monitor and a Photoacoustic Spectroscopy (PAS) monitor.

As further shown in processing block 106, using the PAS monitor comprises providing infrared light from an infrared source, focusing the infrared light with a parabolic mirror in optical communication with the infrared source, using a chopper wheel in optical communication with the parabolic mirror, the chopper wheel producing an alternating sequence of light and an absence of light, using an optical filter in optical communication with the chopper wheel and providing filtered alternating light, using a measurement chamber in optical communication with the optical filter, the measurement chamber having the gaseous sample contained therein; and using at least one microphone, the microphone detecting an acoustic signal resulting from an interaction of the gaseous sample and the filtered alternating light, wherein the acoustic signal correlates to a phosphine level.

Processing block 108 states selectively removing water from said gaseous sample by passing said gaseous sample through an evaporation zone to obtain a evaporated sample. As shown in processing block 110, the evaporation zone comprises a copolymer of tetrafluoroethylene and perfluoro-3,6-dioxa-4-methyl-7-octene-sulfonic acid.

Processing block 112, recites analyzing the evaporated sample in a monitor to determine the amount of phosphine in the evaporated sample. As shown in processing block 114, the analyzing the evaporated sample comprises irradiating the evaporated sample by an infrared source resulting in an irradiated sample, passing the irradiated sample through an optical conduit to a wavelength specific filter, and detecting an energy level of the irradiated sample, wherein the amount of energy in said irradiated sample correlates to a phosphine level. As further shown in processing block 116, the optical conduit includes a gold coating.

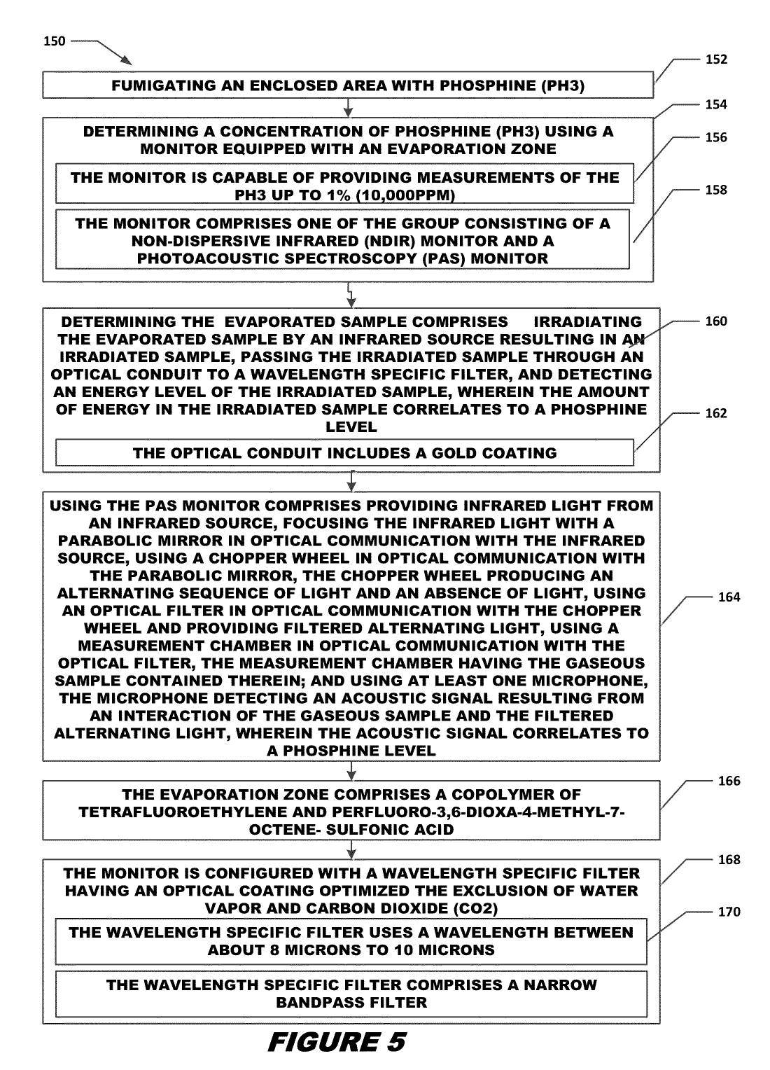

Referring now to FIG. 5, a second embodiment of a method for a Non-Dispersive Infrared Monitor in accordance with a particular embodiment of the present invention is shown. Method 150 begins with processing block 152 which discloses fumigating an enclosed area with phosphine (PH3).

Processing block 154 states determining a concentration of phosphine (PH3) using a monitor equipped with an evaporation zone. Processing block 156 recites wherein the monitor is capable of providing measurements of the PH3 up to 1% (10,000 ppm). Processing block 158 discloses wherein the monitor comprises one of the group consisting of a Non-Dispersive Infrared (NDIR) monitor and a Photoacoustic Spectroscopy (PAS) monitor.

Processing block 160 recites wherein the determining the evaporated sample comprises irradiating the evaporated sample by an infrared source resulting in an irradiated sample, passing the irradiated sample through an optical conduit to a wavelength specific filter, and detecting an energy level of the irradiated sample, wherein the amount of energy in the irradiated sample correlates to a phosphine level.

Processing block 162 discloses wherein said optical conduit includes a gold coating. Processing block 164 states using the PAS monitor comprises providing infrared light from an infrared source, focusing the infrared light with a parabolic mirror in optical communication with the infrared source, using a chopper wheel in optical communication with the parabolic mirror, the chopper wheel producing an alternating sequence of light and an absence of light, using an optical filter in optical communication with the chopper wheel and providing filtered alternating light, using a measurement chamber in optical communication with the optical filter, the measurement chamber having the gaseous sample contained therein; and using at least one microphone, the microphone detecting an acoustic signal resulting from an interaction of the gaseous sample and the filtered alternating light, wherein the acoustic signal correlates to a phosphine level.

As shown in processing block 166 the evaporation zone comprises a copolymer of tetrafluoroethylene and perfluoro-3,6-dioxa-4-methyl-7-octene-sulfonic acid.

Processing block 168 further recites wherein the monitor is configured with a wavelength specific filter having an optical coating optimized the exclusion of water vapor and carbon dioxide (CO2). As shown in processing block 170 the wavelength specific filter uses a wavelength between about 8 microns to 10 microns. And as shown in processing block 172 the wavelength specific filter comprises a narrow bandpass filter.

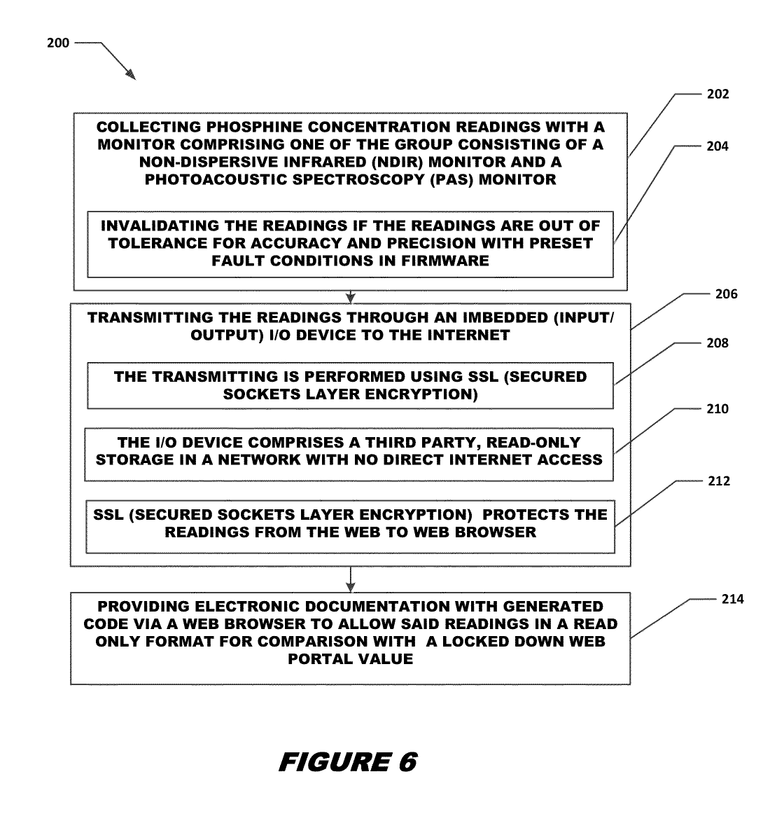

FIG. 6 depicts a first embodiment of a method 200 for providing secured data flow. Comprising. Processing block 202 discloses collecting phosphine concentration readings with a monitor comprising one of the group consisting of a Non-Dispersive Infrared (NDIR) monitor and a Photoacoustic Spectroscopy (PAS) monitor. As shown in processing block 204 the method further includes invalidating the readings if the readings are out of tolerance for accuracy and precision with preset fault conditions in firmware.

Processing block 206 discloses transmitting the readings through an imbedded (Input/Output) I/O device to the Internet. Processing block 208 states wherein the transmitting is performed using SSL (Secured Sockets Layer Encryption). As shown in processing block 210 the I/O device comprises a third party, read-only storage in a network with no direct Internet access. As further shown in processing block 212 SSL (Secured Sockets Layer Encryption) protects said readings from the web to web browser.

Processing block 214 discloses providing electronic documentation with generated code via a web browser to allow said readings in a read only format for comparison with a locked down web portal value.

Throughout the entirety of the present disclosure, use of the articles “a” or “an” to modify a noun may be understood to be used for convenience and to include one, or more than one of the modified noun, unless otherwise specifically stated.

Elements, components, modules, and/or parts thereof that are described and/or otherwise portrayed through the figures to communicate with, be associated with, and/or be based on, something else, may be understood to so communicate, be associated with, and or be based on in a direct and/or indirect manner, unless otherwise stipulated herein.

Although the methods and systems have been described relative to a specific embodiment thereof, they are not so limited. Obviously many modifications and variations may become apparent in light of the above teachings. Many additional changes in the details, materials, and arrangement of parts, herein described and illustrated, may be made by those skilled in the art.

Having described preferred embodiments of the invention it will now become apparent to those of ordinary skill in the art that other embodiments incorporating these concepts may be used. Additionally, the software included as part of the invention may be embodied in a computer program product that includes a computer useable medium. For example, such a computer usable medium can include a readable memory device, such as a hard drive device, a CD-ROM, a DVD-ROM, or a computer diskette, having computer readable program code segments stored thereon. The computer readable medium can also include a communications link, either optical, wired, or wireless, having program code segments carried thereon as digital or analog signals. Accordingly, it is submitted that that the invention should not be limited to the described embodiments but rather should be limited only by the spirit and scope of the appended claims.

What is claimed is:

1. A process for determining the amount of phosphine in the atmosphere of an enclosed area that has been fumigated with phosphine (PH3) the process comprising:
sampling said atmosphere of said enclosed area to obtain a gaseous sample;
selectively removing water from said gaseous sample by passing said gaseous sample through an evaporation zone to obtain a evaporated sample; and
analyzing said evaporated sample in a monitor to determine the amount of phosphine in said evaporated sample.

2. The process of claim 1 wherein said evaporation zone comprises a copolymer of tetrafluoroethylene and perfluoro-3,6-dioxa-4-methyl-7-octene-sulfonic acid.

3. The process of claim 1 wherein said monitor comprises one of the group consisting of a Non-Dispersive Infrared (NDIR) monitor and a Photoacoustic Spectroscopy (PAS) monitor.

4. The method of claim 3 wherein said analyzing said evaporated sample comprises:
irradiating said evaporated sample by an infrared source resulting in an irradiated sample;
passing said irradiated sample through an optical conduit to a wavelength specific filter; and
detecting an energy level of said irradiated sample, wherein the amount of energy in said irradiated sample correlates to a phosphine level.

5. The method of claim 4 wherein said optical conduit includes a gold coating.

6. The method of claim 3 wherein said PAS monitor comprises:
providing infrared light from an infrared source;
a parabolic mirror in optical communication with said infrared source, focusing said infrared light;
a chopper wheel in optical communication with said parabolic mirror, said chopper wheel producing an alternating sequence of light and an absence of light;
an optical filter in optical communication with said chopper wheel and providing filtered alternating light;
a measurement chamber in optical communication with said optical filter, said measurement chamber having said gaseous sample contained therein; and
at least one microphone, said microphone detecting an acoustic signal resulting from said interaction of said gaseous sample and said filtered alternating light, wherein said acoustic signal correlates to a phosphine level.

7. A process comprising:
fumigating an enclosed area with phosphine (PH3);
determining a concentration of phosphine (PH3) using a monitor equipped with an evaporation zone;
wherein said monitor is configured with a wavelength specific filter having an optical coating optimized the exclusion of water vapor and carbon dioxide (CO2).

8. The process of claim 9 wherein said evaporation zone comprises a copolymer of tetrafluoroethylene and perfluoro-3,6-dioxa-4-methyl-7-octene-sulfonic acid.

9. The process of claim 7 wherein said wavelength specific filter uses a wavelength between about 8 microns to 10 microns.

10. The process of claim 7 wherein said wavelength specific filter comprises a narrow bandpass filter.

11. The process of claim 7 wherein said monitor is capable of providing measurements of said PH3 up to 1% (10,000 ppm).

12. The process of claim 7 wherein said monitor comprises one of the group consisting of a Non-Dispersive Infrared (NDIR) monitor and a Photoacoustic Spectroscopy (PAS) monitor.

13. The method of claim 12 wherein said analyzing said evaporated sample comprises:
irradiating said evaporated sample by an infrared source resulting in an irradiated sample;
passing said irradiated sample through an optical conduit to a wavelength specific filter; and
detecting an energy level of said irradiated sample, wherein the amount of energy in said irradiated sample correlates to a phosphine level.

14. The method of claim 13 wherein said optical conduit includes a gold coating.

15. The method of claim 12 wherein said PAS monitor comprises:
providing infrared light from an infrared source;
a parabolic mirror in optical communication with said infrared source, focusing said infrared light;
a chopper wheel in optical communication with said parabolic mirror, said chopper wheel producing an alternating sequence of light and an absence of light;
an optical filter in optical communication with said chopper wheel and providing filtered alternating light;
a measurement chamber in optical communication with said optical filter, said measurement chamber having said gaseous sample contained therein; and
at least one microphone, said microphone detecting an acoustic signal resulting from said interaction of said gaseous sample and said filtered alternating light, wherein said acoustic signal correlates to a phosphine level.

16. A method for providing secured data flow comprising:
collecting phosphine concentration readings with a monitor comprising one of the group consisting of a Non-Dispersive Infrared (NDIR) monitor and a Photoacoustic Spectroscopy (PAS) monitor;
transmitting said readings through an imbedded (Input/Output) I/O device to the Internet; and
providing electronic documentation with generated code via a web browser to allow said readings in a read only format for comparison with a locked down web portal value.

17. The method of claim 16 further comprising invalidating said readings if said readings are out of tolerance for accuracy and precision with preset fault conditions in firmware.

18. The method of claim 16 wherein said transmitting is performed using SSL (Secured Sockets Layer Encryption.

19. The method of claim 16 wherein said I/O device comprises third party, read-only storage in a network with no direct Internet access.

20. The method of claim 16 wherein SSL (Secured Sockets Layer Encryption) protects said readings from the web to web browser.
